(19) United States
(12) Patent Application Publication
(10) Pub. No.: US 2012/0041219 A1
(43) Pub. Date: Feb. 16, 2012

(54) DOUBLE ESTERS AND LUBRICANTS THEREOF

(76) Inventors: Johan A. Thoen, Antwerpen (BE); Jochem Kersbulck, Terneuzen (NL); Jacobus Sinnema, Antwerpen (BE)

(21) Appl. No.: 13/264,249

(22) PCT Filed: Apr. 21, 2009
(86) PCT No.: PCT/US09/41194

§ 371 Date: Oct. 13, 2011

(51) Int. Cl. C07C 55/24; C07C 51/09

(52) U.S. Cl. 554/121

(57) ABSTRACT

A new compound that comprises a branched ester based primarily on renewable resources preferably has good friction properties, a low pour point; acceptable viscosity, and good thermal oxidative stability and is useful as a functional fluid such as a lubricant. The branched ester is a double ester of a polyhydric alcohol having at least two active hydrogen groups separated by at least one carbon atom, wherein each hydroxyl group of the polyhydric alcohol is esterified with one monobasic secondary hydroxy fatty acid group and each hydroxyl of a fatty acid is esterified with one alkyl or cycloalkyl monobasic carboxylic acid group that has from 2 to 22 carbon atoms.

### BACKGROUND

### Field of the Invention

In some aspects, this invention relates to esters of acids derived from natural oils, more specifically to esters of esters of acids derived from natural oils, and their use in lubricants.

Castor oil-based lubricants have an advantage of being based on renewable resources but a disadvantage of having limited thermal oxidative stability because of triglyceride bonds and unsaturated compounds. Furthermore, natural oils (e.g.) castor oil have a high level of linear saturated materials that are detrimental to pour point performance in that, when present in a lubricant or other functional fluid, they reduce its pour point.

Isolated hydroxy-substituted fatty acids or combinations thereof are attainable from natural oils either directly (e.g. ricinoleic acid (12-hydroxyocta-decenoic acid) from castor oil), or by derivation (e.g. production of 12-hydroxy stearic acid from hydroxyoctadecenoic acid). Esters of such acids, particularly esters of 12-hydroxy octadecanoic acid find use in lubricants, but these polyesters are usually linear polyesters or even salts of the acids. Prior art esters are generally linear rather than short chain branched, that is having branches of less than 12 carbon atoms (C₁₂). For instance, each hydroxy fatty acid is reacted with the hydroxyl group of the previous fatty acid in a linear chain. Linear polyesters of this nature have limitations in viscosity, pour point and thermo-oxidative stability. Frequently there are free hydroxyl groups which have the tendency to further react resulting in viscosity increase.

In some aspects, this invention is a composition that comprises a double ester of a polyhydric alcohol, the polyhydric alcohol having at least (≧) two active hydrogen groups separated by at least one carbon atom, wherein each hydroxyl group of the polyhydric alcohol is esterified with a monobasic secondary hydroxy fatty acid group and each hydroxyl group of a fatty acid group is esterified with an alkyl or cycloalkyl monobasic carboxylic acid group having from 2 carbon atoms to 22 carbon atoms. Each monobasic secondary hydroxy fatty acid group and each alkyl or cycloalkyl monobasic fatty acid group independently comes from any compound capable of introducing such a group, for instance a monobasic fatty acid, a monobasic fatty acid anhydride, a monobasic fatty acid chloride or a monobasic fatty acid ester, and is, independently, most preferably 12-hydroxy stearic acid or its methyl ester. The compositions may further comprise one or more of monobasic secondary hydroxy fatty acids and monobasic alkyl or cyclo alkyl carboxylic acids wherein the hydroxyl groups are, on average, reacted with a fatty acid group of the type indicated.

In some aspects, this invention is a process comprising steps of (1) forming a mixture of esters comprising an ester of ≧ one polyhydric alcohol that has at least two active hydrogen groups separated by at least one carbon atom and ≧ one monobasic secondary hydroxy fatty acid or derivative thereof (e.g. a secondary hydroxy fatty acid anhydride, a secondary hydroxy fatty acid halide (e.g. chloride), or a secondary hydroxy fatty acid ester) used in amounts such that each hydroxyl group on the polyhydric alcohol is esterified with (an average of) one acid molecule; and (2) forming an ester of each hydroxyl group of the monobasic secondary hydroxy fatty acid with (an average of) one alkyl or cycloalkyl monobasic carboxylic acid or derivative thereof (e.g. acid anhydride, acid halide or acid ester) having from 2 carbon atoms to 22 carbon atoms. These steps are optionally sequential in either order or are simultaneous or partially simultaneous or concurrent. The same hydroxy fatty acids are preferred for the process as described for the composition. The invention also includes products of this process.

Further, the invention includes compositions including functional fluids comprising compounds of the invention, which fluids are preferably lubricants, power transmission fluids, or heat transfer fluids.

“Friction properties”, as used herein, designates an ability to ameliorate effects of friction between surfaces≧ one of which is moving with respect to the other and can be measured using an instrument designed for such measurements, e.g. PCS Instruments (MTM2 Minitraction Machine) commercially available from PCS Instruments Ltd, London, UK. Preferably measure coefficient of friction, an indicator of friction properties, under conditions of slide-roll-ratio (SRR) 50 percent (%), load 50 newtons (N), speed 100 millimeters per second (mm/sec), and temperature 40 degrees Centigrade (° C.).

“Pour point” is that temperature at which a material solidifies as measured in accord with American Society for Testing and Materials (ASTM) D97.

“Thermal oxidative stability” is resistance to deterioration in the presence of heat (at least 95° C.) and oxygen as measured by the procedure of Deutsche Institut fur Normung (DIN) 51587.

“Double ester” refers to a compound having a polyhydric alcohol esterified with a hydroxy fatty acid that is also esterified at the fatty acid's hydroxyl group. Thus, as the ester of an ester, the compound has two types of ester groups.

“Polyhydric alcohol” designates an organic compound having ≧2 hydroxyl groups.

“Fatty acid” means long-chain carboxylic acids, with chain length of ≧4 carbon atoms. Typical fatty acids have chain length of from 4 carbon atoms to 18 carbon atoms, though some have longer chains. Linear, branched, or cyclic aliphatic groups may be attached to the long chain. Fatty acid residues may be saturated or unsaturated, and they may contain functional groups in addition to the acid group.

“Hydroxy fatty acid”, as used herein, designates a fatty acid having ≧ one hydroxyl group, which is preferably secondary unless stated otherwise. The hydroxyl group is optionally present in the acid as obtained from a natural oil or is introduced by chemical reaction such as by reaction at a double bond, for instance, by epoxidation, reaction with such compounds as maleic anhydride, oxidation, reaction with water such as blown oils where moist air is used in the presence of a catalyst such as cobalt, epoxidation with propylene oxide or higher alkylene oxide, reaction with aqueous perchloric acid and the like. “Monobasic hydroxy fatty acid” is used to designate those fatty acids having one carboxyl group.

All percentages, preferred amounts or measurements, ranges and endpoints thereof herein are inclusive, that is, “less than 10” includes 10 and all real numbers and integers less than (<) 10. “At least” means “greater than or equal to” (“≧”), and “at most” means “less than or equal to” (“≦”). Unless stated otherwise, numbers herein have no more precision than stated. All lists include combinations of two or more members of the list. A range that has an advantageous lower limit combined with a most preferred upper limit is a preferred range for the practice of this invention. All amounts, ratios, proportions and other measurements are by weight unless stated otherwise, implicit from the context, or customary in the art. All percentages refer to weight percent (wt %) based on total composition weight unless stated otherwise, implicit from the context, or customary in the art. Unless stated otherwise or recognized by those skilled in the art as otherwise impossible, steps of processes described herein are optionally carried out in sequences different from the sequence in which the steps are discussed herein. Furthermore, steps optionally occur separately, simultaneously or with overlap in timing.

“Comprising”, is synonymous with “including,” “containing,” or “characterized by,” and is inclusive or open-ended and does not exclude additional, unrecited elements, material, procedures or steps, whether or not the same are disclosed herein. “Consisting essentially of” indicates that in addition to specified elements, materials, procedures or steps; unrecited elements, materials procedures or steps are optionally present in amounts that do not unacceptably materially affect at least one basic and novel characteristic of the subject matter. “Consisting of” indicates that only stated elements, materials, procedures or steps are present except to an extent that has no appreciable effect, thus are substantially absent.

“Or”, unless stated otherwise, refers to listed members individually as well as in any combination of some or all listed members.

Expressions of temperature are optionally in terms either of degrees Fahrenheit (° F.) together with its equivalent in degrees centigrade (° C.) or, more typically, in degrees centigrade (° C.) alone.

The compound of some aspects of the invention is a reaction product of a polyhydric alcohol or mixture of polyhydric alcohols, a monobasic hydroxy fatty acid or mixture of such acids and a monobasic alkyl carboxylic acid or mixture of monobasic carboxylic acids. Compound stoichiometry is such that every hydroxyl group on the polyhydric alcohol is reacted with (at least on average) one monobasic hydroxyl fatty acid molecule, and every hydroxyl group on a fatty acid is reacted with (at least on average) one alkyl monobasic carboxylic acid molecule.

The compound has, at its core, a (molecular moiety from a) polyhydric alcohol or mixture of polyhydric alcohols. Examples of preferred polyhydric alcohols include trimethylolpropane (TMP), pentaerythritol (PE), dipentaerythritol (DPE), neopentyl glycol (NPG) and 2-methyl-2-propyl-1,3-propanediol (MPPD). The polyhydric alcohol is optionally a mixture of polyhydric alcohols. The polyhydric alcohol has an average of ≧2, preferably ≧3, more preferably ≧4, and independently preferably ≦8, more preferably ≦6, and most preferably ≦5 hydroxyl groups. The polyhydric alcohol preferably has an average equivalent weight of ≧24 Daltons (D), preferably ≧30 D, more preferably ≧52 D, and independently preferably ≦100D, more preferably ≦90D, and most preferably ≦80 D.

The polyhydric alcohol is reacted with a monobasic hydroxy fatty acid, which acid may be of vegetable or animal origin. The monobasic hydroxy fatty acid is preferably 12-Hydroxy stearic acid (12-hydroxy octadecanoic acid) as it is a readily available, saturated monohydroxy acid resulting from, for example, hydrogenation of ricinoleic acid obtained from castor oil. Other fatty acids having a secondary hydroxyl group are also useful. Large concentrations of hydroxy fatty acids can also be found in, for instance, Lesquerella oil. Most species in this genus have several hydroxy fatty acids, howewever, they are generally rich in only one. Typical hydroxy fatty acids found in lesquerella oil include: ricinoleic acid(12-hydroxy-9-cis-octadecenoic acid), densipolic acid(12-hydroxy-cis-9,cis-15-octadecadienoic acid), lesquerolic acid(14-hydroxy-cis-11-eicosenoic acid) and auricolic(14-hydroxy-cis-11-cis-17-eicosenoic acid). See Kleiman, R. 1990. “Chemistry of New Industrial Oilseed Crops” p. 196-203, in: J. Janick and J. E. Simon (eds.), Advances in new crops, Timber Press, Portland, Oreg.

Each hydroxyl group on a fatty acid is further esterified using a monobasic alkyl carboxylic acid or a derivative thereof, preferably an anhydride or ester thereof. The monobasic alkyl carboxylic acid has an alkyl group that is linear, branched or cyclic. The acid preferably has ≧2 carbon atoms, more preferably ≧4 carbon atoms, most preferably ≧8 carbon atoms, and independently preferably ≦22 carbon atoms, more preferably ≦16 carbon atoms, and most preferably ≦12 carbon atoms. It has one carboxylic acid group. Examples of the monobasic alkyl carboxylic acid include acetic acid, propionic acid, butyric acid, pentanoic, hexanoic, 2-ethyl hexanoic, valeric acid, caproic acid, caprylic acid, lauric acid and combinations thereof. Preferred alkyl monobasic carboxylic acids include acetic acid, propionic acid, butyric acid, iso-butyric acid, pentanoic acid, hexanoic acid, 2-ethyl-hexanoic acid, capric acid, lauric acid and combinations thereof. More preferred alkyl monobasic carboxylic acids include acetic acid, propionic acid, butyric acid, pentanoic, hexanoic, 2-ethyl hexanoic and combinations thereof. The acids are preferably used in the form of acids, esters or anhydrides, preferably acids or anhydrides, more preferably anhydrides.

The alkyl monobasic carboxylic acids, hydroxyl fatty acids and their esters, particularly 12-hydroxyl stearic acid and its methyl ester, and polyhydric alcohols are commercially available.

In esterification, the secondary hydroxy fatty acid or its anhydride and the polyhydric alcohol, the hydroxy fatty acid and the carboxylic acid or its anhydride, or all three reactants are contacted, preferably in the presence of at least one acidic or basic catalyst, under reaction conditions (conditions effective to result in formation of an ester between the acid and hydroxyl groups). Such conditions include a temperature ≧150° C., preferably ≧170° C., more preferably ≧190° C. independently to preferably ≦240° C., more preferably ≦220° C., most preferably ≦200° C. at atmospheric pressure. The pressure is conveniently atmospheric pressure, but lower pressures are also useful, for instance, the pressure is preferably ≧50 hectopascals (hPa), preferably ≧30 hPa, more preferably from ≧10 hPa independently to preferably ≦200 hPa, more preferably ≦150 hPa, most preferably ≦100 hPa. The amount of catalyst is preferably ≧100 ppm, preferably ≧200 ppm, more preferably ≧400 ppm independently to preferably ≦3000 ppm, more preferably ≦2000 ppm, most preferably ≦1000 ppm (parts per million by weight) based on total weight of reactants. Reaction conditions preferably include stirring sufficient to effect contact among the reactants. Reaction time depends on such variables as temperature, pressure, type of catalyst and catalyst concentration. In most instances, the time is ≧240 minutes to ≦90 hours. Preferably, the reaction time is ≧500 minutes, more preferably ≧450 minutes, more preferably ≧7 hour to preferably ≦90 hours, more preferably ≦40 hours and most preferably ≦20 hours. Hydrocarbons are optionally used as entrainment agents to facilitate the removal of volatile components from a (trans)esterification reaction. Useful hydrocarbons include aliphatic and aromatic hydrocarbons such as iso-octane, toluene, xylene and the like and combinations thereof.

For transesterification, at least one of the secondary hydroxy fatty acid, alkyl monobasic carboxylic acid or both are in the form of their esters for reaction with a corresponding hydroxyl group. Reactant contact preferably occurs in the presence of a transesterification catalyst and under reaction conditions. Such conditions include a temperature ≧130° C., preferably ≧140° C., more preferably ≧150° C. independently to preferably ≦240° C., more preferably ≦220° C., most preferably ≦200° C. at atmospheric pressure. The pressure is conveniently atmospheric pressure, but lower pressures are also useful, for instance, the pressure is preferably ≧50 hectopascals (hPa), preferably ≧30 hPa, more preferably ≧10 hPa independently to preferably ≦200 hPa, more preferably ≦150 hPa, most preferably ≦100 hPa. The catalyst is any catalyst or combination thereof known to skilled artisans for use in catalyzing transesterification. Such catalysts include organometallic catalysts that contain tin, titanium, zinc, or cobalt, carbonate catalysts (e.g., potassium carbonate (K₂CO₃), sodium carbonate (NaHCO₃), and lithium carbonate (LiCO₃)), other bases (e.g., sodium hydroxide (NaOH) or potassium hydroxide (KOH)), or a combination thereof. Organometallic catalysts, particularly those of tin and titanium are preferred. Lithium carbonate is a preferred carbonate. Reaction time depends on such variables as temperature, pressure, type of catalyst and catalyst concentration. In most instances, the time is ≧240 minutes to ≦90 hours. Preferably, the reaction time is ≧500 minutes, more preferably ≧450 minutes, more preferably ≧7 hour to preferably ≦90 hours, more preferably ≦40 hours and most preferably ≦25 hours. Hydrocarbons are optionally used as entrainment agents to facilitate the removal of volatile components from a (trans)esterification reaction. Useful hydrocarbons include aliphatic and aromatic hydrocarbons such as iso-octane, nonane, toluene, xylene and the like and combinations thereof.

The amount of catalyst is preferably at least enough to effect a reaction between the acid ester and hydroxyl group to form the resulting ester at a desirable rate. The amount of catalyst depends, for example, on the particular type of catalyst and reactants. When a tin, titanium or carbonate catalyst is employed, the amount of catalyst is advantageously ≧100 ppm, preferably ≧250 ppm, more preferably ≧500 ppm, and most preferably ≧1000 ppm based on total weight of reactants. Considerations other than operability determine any preference for upper limits. While more is operable, even suitable, such considerations as cost of the catalyst or necessity of deactivating or removing excess indicate that, in most cases, the amount of catalyst is preferably ≦10000 ppm, more preferably ≦8000 ppm, most preferably ≦6000 ppm, based on total weight of reactants. The tin catalyst can be any known tin transesterification catalyst such as tin (II) octanoate, tin (II) 2-ethylheptanoate, dibutyl tin (IV) dilaurate, and other tin catalysts that are similarly functionalized. Preferably the tin catalyst is tin (II) octanoate, tin (II) 2-ethylheptanoate, dibutyl tin (IV) dilaurate, or a combination thereof. The titanium catalyst can be any titanium catalyst known to be effective for transesterification, such as tetra-n-butyl titanate, titanium tetraisopropoxide, titanium tetraisobutoxide, or any appropriately functionalized titanium (IV) alkoxide. Tetra-n-butyl titanate is a preferred titanium catalyst.

In both esterification and transesterification, one typically starts a reaction at or slightly above a preferred temperature range's lower limit, maintains that temperature for a portion of the reaction time (e.g. 120 minutes, 180 minutes or 240 minutes or, alternatively, until one attains a desired level (e.g. 25%) of ester formation) and then increases temperature to a higher temperature within a preferred temperature range. One may also add any reactant or combination of reactants in incremental portions, especially if alternative undesirable reactions are possible under reaction conditions.

In both esterification and transesterification, preferably use near stoichiometric amounts of reactants to achieve desired end products. Especially in reacting a polyhydric alcohol and a secondary hydroxy fatty acid, avoid a large excess of hydroxy fatty acid to minimize, preferably eliminate, formation of hydroxy fatty acid chains. A preferred equivalent molar ratio of secondary hydroxy fatty acid to hydroxyl groups on the polyhydric alcohol is ≦1/1, and independently preferably ≧0.6/1, more preferably ≧0.83/1. A preferred equivalent molar ratio of the monobasic alkyl carboxylic acid (also known as a “capping agent”) to unreacted hydroxyl groups is preferably ≧1/1, more preferably ≧1.01/1 to avoid presence of free hydroxyl groups in the final product, and independently preferably ≦2.0/1, most preferably ≦1.1/1.

In a preferred embodiment, react the secondary hydroxy fatty acid, polyhydric alcohol and alkyl monobasic carboxylic acid reactants in one reaction, or at least one pot, rather than in separate reactions, e.g., by mixing the reactants, adding the catalyst, heating the reactants and catalyst to a temperature of, for example, 140° C. to initiate reaction among the reactants, and then stepwise increasing the temperature over a period of 240 minutes to a temperature within a range of from 180° C. to 200° C.

When the secondary monobasic hydroxy fatty acid is 12-hydroxyl stearic acid, and the polyhydric alcohol is a diol, the resulting composition comprises a double ester structure conveniently represented as:

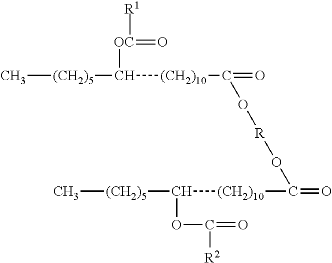

wherein —O—R—O— corresponds to the polyhydric alcohol, thus R is any alkyl, alkenyl, alicyclic or heterocyclic moiety, preferably of ≧6 carbon atoms, more preferably ≧7 carbon atoms, and independently preferably ≦12 carbon atoms, more preferably ≦10 carbon atoms, and most preferably ≦8 carbon atoms; R1 and R2 are optionally the same or different and are each the alkyl groups of an alkyl monobasic carboxylic acid capping agent, and thus are linear, branched or cyclic alkyl groups preferably ≧2 carbon atoms, more preferably ≧4 carbon atoms, and independently preferably ≦22 carbon atoms, more preferably ≦16 carbon atoms, and most preferably ≦12 carbon atoms. The above structural representation evidences a branched structure for the double ester product.

The resulting double esters are especially useful in functional fluids, which fluids are preferably lubricants, power transmission fluids, heat transfer fluids, or metal working fluids.

The double ester preferably has at least one of a) friction properties as indicated by a coefficient of friction (as measured using a PCS Instruments (MTM2 Minitraction Machine) commercially available from PCS Instruments Ltd, UK under conditions of: slide-roll-ratio (SRR): 50 percent, load 50 N, speed 100 mm/sec, and temperature, 40° C.) of preferably ≦0.07, more preferably ≦0.06, most preferably ≦0.05; b) a pour point temperature (ASTM D97) ≦0° C., preferably ≦−5° C.; c) a viscosity (ASTM D445-94) that is preferably ≦200 centistokes (cSt), more preferably ≦150 cSt, most preferably ≦100 cSt at a temperature of 40° C.; or d) a thermal oxidative stability (DIN 51587) that is preferably ≦5 milligrams of potassium hydroxide per gram of double ester (mg KOH/g), more preferably ≦2 mg KOH/g. Use standard tests to measure these properties.

Examples (Ex), represented as Arabic Numerals, that follow illustrate, but do not limit, various aspects of this invention.

### EXAMPLES

### Example 1

### Formation of an Acetyl Double Ester of Neopentyl Glycol Starting with 12-Hydroxy Stearic Acid

In a reaction flask, acetylate 12-hydroxy stearic acid (12-HSA) by refluxing 600 parts by weight (pbw) of 12-HSA with 192 pbw of acetic anhydride for one hour at 160° C. at atmospheric pressure. After that time, reduce the pressure to 20 hPa, and remove a major proportion of acetic acid and unreacted acetic anhydride via distillation to yield an ester (12-acetoxy stearic acid). Deodorize the ester by sparging with steam under vacuum to free it from remaining traces of acetic anhydride or acid. Esterify the ester by reacting 600 pbw of 12-acetoxy stearic acid with 114 pbw of neopentylglycol (NPG) at a temperature of 160° C. Remove water resulting from the reaction via distillation, collecting a total of 26 pbw of water from the reaction flask over a period of 7 hours. Heat contents of the reaction flask to 200° C. and reduce the pressure 10 hPa to remove excess NPG. Cool flask contents to room temperature, nominally 25° C., to yield a double ester product present as a viscous liquid with a viscosity of 146 cSt at 40° C.

### Example 2

### Formation of an Acetyl Double Ester of Neopentyl Glycol Starting with 12-Hydroxy Stearic Acid

Esterify 12-Hydroxystearic acid (12-HSA) by reacting 600 grams (g) of 12-HSA with 114 g of NPG for a period of seven hours, gradually increasing temperature from 150° C. to 180° C. during the first three hours, then reducing the temperature to 160° C. for the remaining four hours, collecting a total of 36 g of water during the seven hours. Acetylate 500 g of the 12-HSA-NPG ester with 168 g of acetic anhydride over a two hour period at 125° C., then remove acetic acid and excess acetic anhydride via distillation at a temperature of 160° C. and a pressure of 20 hPa. The double ester product is a viscous liquid with a viscosity of 110 cSt at 40° C. and a pour point of −11° C.

### Example 3

### Formation of an Acetyl Double Ester of Neopentyl Glycol Starting with 12-Hydroxy Stearic Acid Methyl Ester, Applying a Catalyst

Esterify 12-Hydroxystearic acid methyl ester (12-HSAME) with NPG, by reacting 628 g of 12-HSAME with 104 g NPG for a period 6 hours in the presence of 200 ppm tetraisopropylorthotitanate (TIPT) catalyst, at a temperature of 180° C., collecting 64 g of methanol during said period. Cool the reaction mixture to 125° C. and add 183 g of acetic anhydride. Continue the reaction, an acetylation reaction, for a period of 4 hours, removing 108 g of acetic acid during said period. Increase the temperature to 160° C. and reduce the pressure to 20 hPa to remove the remaining acetic acid. The resulting liquid has a viscosity of 61 cSt at 40° C., and a pour point of −11 C.

### Example 4

### Formation of an Isobutoyl Double Ester of Neopentyl Glycol Starting with Methyl-12-Hydroxy Stearate, Applying a Catalyst

In a 1.1 liter (L) glass reactor equipped with temperature controllers, an overhead stirrer, an electric heater and a Dean-Stark apparatus with a water condenser connected to a vacuum/nitrogen line, place 699.5 g (2.2 moles) of 12-HSAME, 140.0 g of NPG, 123.3 g of nonane (as an entrainment liquid) and 4.57 g (0.005 mole) tin dioctoate (Sn(II)dioctoate) catalyst. Place a Vigreux distillation column between the reactor and the Dean-Stark apparatus to prevent NPG from distilling off with methanol. Heat reactor contents to 155° C. and remove methanol via azeotropic distillation with the nonane, collecting 68 g of methane after 20 hours of heating.

Remove remaining nonane under reduced pressure, then cool reactor contents to 120° C. before adding 424.0 g (2.68 moles) of isobutyric anhydride to the reactor as a capping reactant. After two hours, remove unreacted anhydride and acid formed during capping under reduced pressure, maintaining the reduced pressure for two hours at a temperature of 160° C. maximize acid removal.

Cool reactor contents 70° C. and add 100 milliliters (mL) of aqueous one molar (1M) sodium carbonate (NaHCO3) solution to the contents with stirring. Continue stirring for one hour, then remove water from the reactor under reduced pressure (10 hPa?).

Add 10 g of magnesium sulfate (MgSO₄), 10 g of magnesium silicate, and 5 g of activated carbon to the reactor, then filter the reactor contents using a filter paper pre-coated with 809 g of magnesium silicate. The resulting liquid has a viscosity of 57.6 cSt at 40° C., and a pour point of −13 C.

1. A composition that comprises a double ester of a polyhydric alcohol, the polyhydric alcohol having at least two active hydrogen groups separated by at least one carbon atom, wherein each hydroxyl group of the polyhydric alcohol is esterified with a monobasic secondary hydroxy fatty acid group and each hydroxyl group of a fatty acid is esterified with a monobasic alkyl or monobasic cycloalkyl carboxylic acid group having from 2 to 22 carbon atoms.

2. The composition of claim 1 wherein 12-hydroxy stearic acid or 12-hydroxy stearic acid methyl ester serves as a source for the monobasic secondary hydroxy fatty acid group.

3. The composition of claim 1 wherein a fatty acid or fatty acid derivative of Lesquerella oil serves as a source for the secondary monobasic hydroxy fatty acid group.

4. The composition of claim 1 wherein the polyhydric alcohol has an average of at least 2, and at most 8 hydroxyl groups and an average equivalent weight of from 24 to 100 Daltons.

5. The composition of wherein the polyhydric alcohol is selected from trimethylolpropane (TMP), pentaerythritol (PE), dipentaerythritol (DPE), neopentyl glycol (NPG) and 2-methyl-2-propyl-1,3-propanediol (MPPD) or a combination thereof.

6. The composition of claim 1 wherein a member selected from a group consisting of acetic acid, propionic acid, butyric acid, pentanoic, hexanoic, 2-ethyl hexanoic, valeric acid, caproic acid, caprylic acid, lauric acid or a halide, ester or anhydride of any such acid serves as a source for the monobasic alkyl carboxylic acid group.

7. The composition of claim 1 wherein the double ester has at least one of (a) friction properties as indicated by a coefficient of friction of at most about 0.07; (b) a pour point of less than or equal to 0° C.; (c) a viscosity of less than or equal to 200 centistokes at a temperature of 40° Centigrade; or (d) a thermal oxidative stability of less than or equal to 5 milligrams potassium hydroxide per gram of double ester.

8. A functional fluid comprising at least one composition of claim 1.

9. The functional fluid of claim 8 wherein the functional fluid is a lubricant, power transmission fluid, heat transfer fluid, metal working fluid, or combination thereof.

10. A process comprising steps of (1) forming a mixture of esters comprising an ester of at least one polyhydric alcohol that has at least two active hydrogen groups separated by at least one carbon atom and at least one monobasic secondary hydroxy fatty acid or derivative thereof used in amounts such that each hydroxyl group on the polyhydric alcohol is esterified with an average of one fatty acid molecule; and (2) forming an ester of each hydroxyl group of the monobasic fatty acid group with an average of one an alkyl or cycloalkyl monobasic carboxylic acid or derivative thereof that has from 2 carbon atoms to 22 carbon atoms.

11. The process of claim 10, wherein step (1) employs a catalyst selected from a group consisting of organometallic catalysts that contain tin, titanium, zinc or cobalt.
